SEQUENCE LISTING (38 sequences)

SEQ ID NO: 1 - protein, 3 aa, Artificial Sequence
  As derived from human thought based in part on observation and experiment
WSQ

SEQ ID NO: 2 - protein, 3 aa, Artificial Sequence
  As derived from human thought based in part on observation and experiment
WSL

SEQ ID NO: 3 - protein, 3 aa, Artificial Sequence
  As derived from human thought based in part on observation and experiment
WQY

SEQ ID NO: 4 - protein, 3 aa, Artificial Sequence
  As derived from human thought based in part on observation and experiment
SQY

SEQ ID NO: 5 - protein, 3 aa, Artificial Sequence
  As derived from human thought based in part on observation and experiment
SQL

SEQ ID NO: 6 - protein, 3 aa, Artificial Sequence
  As derived from human thought based in part on observation and experiment
SNY

SEQ ID NO: 7 - protein, 3 aa, Artificial Sequence
  As derived from human thought based in part on observation and experiment
QYL

SEQ ID NO: 8 - protein, 3 aa, Artificial Sequence
  As derived from human thought based in part on observation and experiment
QNY

SEQ ID NO: 9 - protein, 3 aa, Artificial Sequence
  As derived from human thought based in part on observation and experiment
WSL

SEQ ID NO: 10 - protein, 3 aa, Artificial Sequence
  As derived from human thought based in part on observation and experiment
SNY

SEQ ID NO: 11 - protein, 3 aa, Artificial Sequence
  As derived from human thought based in part on observation and experiment
WQQ

SEQ ID NO: 12 - protein, 3 aa, Artificial Sequence
  As derived from human thought based in part on observation and experiment
WNQ

SEQ ID NO: 13 - protein, 3 aa, Artificial Sequence
  As derived from human thought based in part on observation and experiment
WYQ

SEQ ID NO: 14 - protein, 3 aa, Artificial Sequence
  As derived from human thought based in part on observation and experiment
WQL

SEQ ID NO: 15 - protein, 3 aa, Artificial Sequence
  As derived from human thought based in part on observation and experiment
WNL

SEQ ID NO: 16 - protein, 3 aa, Artificial Sequence
  As derived from human thought based in part on observation and experiment
WYL

SEQ ID NO: 17 - protein, 3 aa, Artificial Sequence
  As derived from human thought based in part on observation and experiment
WSY

SEQ ID NO: 18 - protein, 3 aa, Artificial Sequence
  As derived from human thought based in part on observation and experiment
WNY

SEQ ID NO: 19 - protein, 3 aa, Artificial Sequence
  As derived from human thought based in part on observation and experiment
WYY

SEQ ID NO: 20 - protein, 3 aa, Artificial Sequence
  As derived from human thought based in part on observation and experiment
SSQ

SEQ ID NO: 21 - protein, 3 aa, Artificial Sequence
  As derived from human thought based in part on observation and experiment
SQQ

SEQ ID NO: 22 - protein, 3 aa, Artificial Sequence
  As derived from human thought based in part on observation and experiment
SNQ

SEQ ID NO: 23 - protein, 3 aa, Artificial Sequence
  As derived from human thought based in part on observation and experiment
SSL

SEQ ID NO: 24 - protein, 3 aa, Artificial Sequence
  As derived from human thought based in part on observation and experiment
SNL

SEQ ID NO: 25 - protein, 3 aa, Artificial Sequence
  As derived from human thought based in part on observation and experiment
SYL

SEQ ID NO: 26 - protein, 3 aa, Artificial Sequence
  As derived from human thought based in part on observation and experiment
SSY

SEQ ID NO: 27 - protein, 3 aa, Artificial Sequence
  As derived from human thought based in part on observation and experiment
SYQ

SEQ ID NO: 28 - protein, 3 aa, Artificial Sequence
  As derived from human thought based in part on observation and experiment
SYY

SEQ ID NO: 29 - protein, 3 aa, Artificial Sequence
  As derived from human thought based in part on observation and experiment
QSQ

SEQ ID NO: 30 - protein, 3 aa, Artificial Sequence
  As derived from human thought based in part on observation and experiment
QQQ

SEQ ID NO: 31 - protein, 3 aa, Artificial Sequence
  As derived from human thought based in part on observation and experiment
QNQ

SEQ ID NO: 32 - protein, 3 aa, Artificial Sequence
  As derived from human thought based in part on observation and experiment
QYQ

SEQ ID NO: 33 - protein, 3 aa, Artificial Sequence
  As derived from human thought based in part on observation and experiment
QSL

SEQ ID NO: 34 - protein, 3 aa, Artificial Sequence
  As derived from human thought based in part on observation and experiment
QQL

SEQ ID NO: 35 - protein, 3 aa, Artificial Sequence
  As derived from human thought based in part on observation and experiment
QNL

SEQ ID NO: 36 - protein, 3 aa, Artificial Sequence
  As derived from human thought based in part on observation and experiment
QSY

SEQ ID NO: 37 - protein, 3 aa, Artificial Sequence
  As derived from human thought based in part on observation and experiment
QQY

SEQ ID NO: 38 - protein, 3 aa, Artificial Sequence
  As derived from human thought based in part on observation and experiment
QYY

(19) United States
(12) Patent Application Publication
(10) Pub. No.: US 2012/0010158 A1
(43) Pub. Date: Jan. 12, 2012

(54) TUMOR NECROSIS FACTOR INHIBITING PEPTIDES AND USES THEREOF

(75) Inventors: Rajesh Jain, New Delhi (IN); Virendra Kumar Vinayak, New Delhi (IN); Shweta Dubey, New Delhi (IN); Shudhanand Prasad, New Delhi (IN); Vijay Goel, New Delhi (IN); Rahul Jain, New Delhi (IN)

(73) Assignee: PANACEA BIOTEC LTD., New Delhi (IN)

(21) Appl. No.: 13/130,267

(22) PCT Filed: Nov. 5, 2009
(86) PCT No.: PCT/IN2009/000626

§ 371 Date: May 19, 2011

(30) Foreign Application Priority Data
Nov. 20, 2008 (IN) 2622/DEL/2008

(51) Int. Cl. A61K 38/06; C07K 5/083; A61P 1/00; A61P 19/02; A61P 17/06; C07K 5/08; A61P 37/06

(52) U.S. Cl. 514/21.9; 530/331

(57) ABSTRACT

The present invention relates to Tumor Necrosis Factor-alpha (TNF-alpha or TNF-α) inhibiting peptides and process for the preparation thereof. The present invention further relates to a pharmaceutical composition comprising TNF-alpha inhibiting peptides of the present invention and uses thereof in treating TNF-alpha mediated inflammatory disorders.

### FIELD OF THE INVENTION

The present invention relates to biologically active peptides and process for the preparation thereof. The present invention further relates to Tumor Necrosis Factor-alpha (TNF-α or TNF-alpha) inhibiting peptides and proceMss for the preparation thereof. The present invention further relates to a pharmaceutical composition comprising said peptide molecules and uses thereof in treating Tumor Necrosis Factor-alpha (TNF-α or TNF-alpha) mediated inflammatory disorder such as rheumatoid arthritis, psoriatic arthritis, Crohn's disease, and sepsis etc.

### BACKGROUND OF THE INVENTION

Cytokines are a class of signaling proteins produced by activated immune cells i.e. B cells, T cells and monocytes and macrophages. The cytokines include family of interleukins (IL-1 through IL-23), Interferons (alpha, beta and gamma) and TNF-α and β (Janeway Calif. et al. 1999, Immunobiology, 4^{th} Ed. N.Y., Garland, 1999; Roitt I et al. 2002, Immunology 5^{th} ed. London, Mosby, 2002). Important roles for IL-1 and TNF-α as key mediators of inflammatory response in diseases like sepsis and autoimmune conditions like RA, dermatological disorders, inflammatory bowel disease has been suggested (Locksley RM et al. 2001, Cell 104(4):487-501, Feldmann M et al 1996, Ann. Rev. Immunol. 14: 397-440; Buchan G et al. 1988, Clin. Exp. Immunol. 73:449-455; Canto E et al. 2006, Clin. Immunol. 119(2):156-165; Fantuzzi F et al. 2008, Expert Opi. Ther. Targets 12(9):1085-96). Cytokine regulation studies have further suggested that TNF-α is the most important cytokine responsible for inflammatory pathology (Feldmann et al, 1999, Ann. Rheum. Dis. 58: (Suppl 1) 127-131).

TNF-alpha was originally discovered as a molecule which caused hemorrhagic necrosis of mouse tumors (Carswell et al., 1975, Proc. Natl. Acad. Sci. U.S.A. 72:3666). A second line of investigation of a serum protein known as “cachectin”, thought to be responsible for the condition of cachexia, led to the eventual discovery that cachectin was identical to TNF-alpha (Beutler et al., 1989, Annu. Rev. Immunol. 7:625). TNF-alpha has now been established as a broadly active inflammatory mediator involved in diverse clinical conditions.

TNF-alpha is a 17 kD molecular weight protein produced by several cell types, particularly activated macrophages. TNF-alpha is initially synthesized as a transmembrane protein arranged in stable trimers. This is subsequently cleaved by metalloprotease-TNF alpha converting enzyme (TACE) to form the homotrimeric soluble TNF (sTNF) which engages to its cognate receptors (TNFRI, p55 and TNFRII, p75), expressed ubiquitously. The ubiquitous expression of TNF receptors along with cell specific effectors explains wide variety of TNF-α mediated cellular response, some of which are deleterious and life threatening. These receptors when shed from mononuclear cells, lower the TNF-α levels by mopping up and acting as natural inhibitors.

TNF-alpha induces a wide variety of cellular responses, many of which result in deleterious consequences. For example, TNF-alpha induces cachexia which is a condition resulting from loss of fat and whole body protein depletion, accompanied by insufficient food intake due to anorexia. Cachexia is commonly seen in cancer patients, and it has also been observed in patients with acquired immunodeficiency syndrome (AIDS). In addition, injection of high doses of TNF-alpha in animals produces most of the symptoms of septic shock. TNF-alpha has also been shown to play a role in autoimmune diseases such as multiple sclerosis and rheumatoid arthritis, psoriasis, psoriatic arthritis, hypersensitivity, immune complex diseases and graft versus host disease as well as transplantation rejection. The involvement of TNF-alpha has even been implicated in malaria and lung fibrosis. Therefore it is of considerable interest and therapeutic benefit to target blocking of TNF-alpha production in disease conditions.

### Treatment of TNF-Associated Disorders

Methods for neutralizing the adverse effects of TNF-alpha have focused on the use of anti-TNF antibodies and soluble TNF-R. In animal models, treatment of TNF-alpha associated inflammatory disorders with antibodies specific for TNF-alpha has shown therapeutic efficacy (Williams et al., 1992, Proc. Natl. Acad. Sci. U.S.A. 89:9784-88; Baker et al., 1994, Eur. J. Immunol. 24:2040; Suitters et al., 1994, J. Exp. Med. 179:849). Chimeric forms of anti-TNF antibodies have been constructed for use in human clinical trials (Lorenz et al., 1996, J. Immunol. 156:1646; Walker et al., 1996, J. Infect. Dis. 174:63; Tak et al, 1996, Arthritis Rheumat. 39:1077). Additionally, soluble TNF-receptor fusion proteins have been introduced as TNF-antagonists in human patients (Peppel et al., 1991, J. Exp. Med. 174:1483; Williams et al., 1995, Immunol. 84:433; Baumgartner et al., 1996, Arthritis Rheumat. 39 (Suppl.) S74).

U.S. Pat. No. 6,265,535 relates to the cyclic peptides and peptide analogues designed from a binding loop of a TNF-receptor superfamily member which interfere with the binding interactions between TNF-alpha and TNF-receptor, exhibiting inhibitory activities in-vitro as well as in-vivo, to antagonize the undesirable biological activities of TNF in vivo. This invention prefers cyclized peptides since loops and turns in play functionally important roles in protein-protein interactions. In specific embodiments cyclic peptides have been designed from three binding loops of TNF-R p55 which bind with TNF-α and inhibit the binding of TNF-α to its cellular receptors. Most preferred embodiments have atleast 7 amino acids and there is no suggestion that smaller linear peptides could be useful as TNF alpha inhibitors.

U.S. Pat. No. 6,344,443 relates to a method for inhibiting TNF-alpha binding to TNF receptors and TNF-α function by administering an effective amount of an inhibitory peptide. The patent relates to the peptides which bind to TNF receptors and interfere with the ability of TNF-a to bind to and activate cellular TNF-α receptors. Particularly, this invention relates to the use of peptides having 7 and 12 aminoacid residues for inhibiting TNF-alpha binding to TNF receptors and TNF function. The '443 document aims at screening small peptides that could bind to TNF receptors. The smallest molecule that has been identified is 7 amino acid long sequence. There is no suggestion that further smaller peptides could be useful as TNF alpha inhibitors.

U.S. Pat. No. 6,143,866 relates to a urine derived TNF inhibitor peptide. The patent further relates to purified forms of TNF inhibitor which are active against TNF alpha. The patent further relates to purified forms of TNF inhibitor which would be valuable as pharmaceutical preparations exhibiting activity against TNF. It further relates to 30 kDa protein and a 40 kDa protein which have been obtained in their purified forms. The amino acid sequences disclosed for these proteins is not less than 15 amino acids long, hence there is no suggestion that further smaller peptides could be useful as TNF alpha inhibitors.

U.S. Pat. No. 6,048,543 relates to the use of at least one amino acid selected from the group consisting of glycine, alanine and serine, or the physiologically acceptable salts thereof, in the preparation of a medicament or nutritional formulation for the diminution of tumor necrosis factor (TNF) levels in patients in whom said levels are elevated beyond those which mediate physiological homeostasis and local inflammation. The patent discloses that the diminution of TNF levels can be achieved by inhibition or diminution of, TNF production by macrophage-type cells or TNF release by macrophage type cells or binding of TNF by TNF receptors. The patent relates to the use of only glycine, alanine and serine in the preparation of a medicament or nutritional formulation for diminution of TNF levels. The patent does not disclose any use of combination of aminoacids or peptides containing combination of aminoacids.

U.S. Pat. No. 6,107,273 relates to TNF-α antagonist compounds that comprise a molecular surface that is substantially similar to at least one molecular surface of human TNF-α selected from the group of molecular surfaces of human TNF-α. The compounds of the invention have linking moiety attached at both the ends and a spacer moiety. The compound of the invention discloses the TNF-alpha inhibiting peptides having 25 aminoacids along with a linking moiety and a spacer moiety. The TNF-α antagonist compounds of the '273 patent binds to TNF p55 receptor and/or TNF p75 receptor and inhibit TNF-α mediated cytotoxicity. The '273 patent does not suggest the smaller peptides without the spacer moieties.

The therapeutics currently available in this area has been designed to neutralize TNF-α by using soluble TNF receptors or monoclonal anti-TNF antibodies (Piguet et al. 1992, Immunology 77:510-514; Elliot et al. 1993, Arthritis Rheum. 36: 1681-90). These bind to circulating TNF-a thereby limiting latter's accessibility to TNF-R on cell surface and subsequent activation of inflammatory pathways. The available therapies to block TNF-alpha levels are (1) Infliximab (Remicade): a mouse human chimeric anti-human TNF-alpha monoclonal antibody (2) Humira: a fully human anti TNF-alpha monoclonal antibody (3) Etanercept, a dimeric fusion protein of soluble P75sTNF-RII fused to Fc portion of human IgG. Although these molecules have shown efficacy in various autoimmune disorders but these suffer from the certain limitations namely, poor bioavailability, stability, induction of severe immune reactions and high costs. It would be pertinent to provide alternative means to block TNF-alpha activity.

Sequences of anti-TNF antibodies or TNF receptors have been utilized for synthesis of biologically active peptide fragments (Weisong Q et al 2006, Mol. Immunol. 43:660-66; Zhang J et al. 2003, Biochem. Biophy. Res. Comm. 7:1181-87; Aoki et al. 2006, J. Clin. Invest. 116(6): 1525-34); but these are large and relatively insoluble. This highlights the need of an improved TNF-alpha inhibitor compound possibly in form of a small molecule.

In one such study, Takasaki et al, 1997 (Nat. Biotechnol. 1997, November; 15(12):1266-70) studied peptide analogues designed from three binding loops of TNF-RI. Such peptides were generated based on the amino acid sequences that form the binding loops. One of the peptide sequences cyclic WP9 (sequence WSENL) on loop 1 of domain 3 on TNF-RI (residues 107-114) was found to be most promising for TNF-alpha blocking activity. The sequence WSENL of WP9 was used as template by Takasaki et al to design cyclic peptidomimeticsWP9Q, WP9ELY, WP9Y, and WP9QY. Peptidomimetic WP9QY showed therapeutic values and reduced the severity of experimental autoimmune encephalomyelitis (EAE) & Rheumatoid arthritis (RA) in mice. However, its relatively poor solubility in physiological buffers is a limiting factor in its use as a potential human therapeutic (Takasaki et al. 1997, Nat. Biotechnol. Nov. 15(12):1266-70). Although, cyclization and aromatization of a peptide enhanced stability and bioavailability yet little or no effect on enhanced solubility or enhanced activity was noticed by Takasaki et al, 1997. Takasaki stated that WP9QY is the smallest peptidomimetic developed till date and it might be used as a lead compound for the next generation of non peptidic inhibitors. Hence this reference suggests a person skilled in the art to try non-peptidic TNF inhibitors based on the smallest known peptide known at that time.

Such observation underscores the need to develop an improved next generation TNF-α inhibitors aimed at designing a peptide having solubility in physiological buffer, better stability and bioavailability with least side effects and reduced cost.

### SUMMARY OF THE INVENTION

According to the present invention there is provided a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids.

According to another aspect of the present invention there is provided a TNF-α inhibiting peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids.

The present invention further relates to the use of biologically active peptides of the present invention for the treatment of TNF-α related disease conditions. The present invention also relates to a pharmaceutical composition comprising biologically active peptides of the present invention.

### BRIEF DESCRIPTION OF DRAWINGS

FIG. 1 shows Anti-TNF-α activity of the peptides employing L929 bioassay. Numbers 1-10 on X-axis denotes the peptides of Sequence ID-1 to 10. Y axis denotes the Mean±SE percentage inhibition of TNF-alpha mediated cytotoxicity of three independent experiments, each run in duplicate. On the X axis, ‘+ve’ denotes positive control i.e. a known TNF-alpha inhibitory peptide i.e. Cyclic WSQYL (cy Trp-Ser-Gln-Tyr-Leu).

FIG. 2 shows comparison of linear peptide of Sequence ID-2 and Sequence ID-6; cyclic peptide of Sequence ID-9 and Sequence ID-10 and positive control i.e. Cyclic WSQYL (cy Trp-Ser-Gln-Tyr-Leu) peptide to inhibit TNF-α induced cytotoxicity. Results have been expressed as Mean±SE percentage inhibition of TNF-alpha mediated cytotoxicity of three independent experiments, each run in triplicate.

FIG. 3 shows comparison of inhibition of TNF-alpha mediated cytotoxicity by peptide of sequence ID-6 and Etanercept (Et). Results have been expressed as Mean±SE percentage inhibition of TNF-alpha mediated cytotoxicity of two independent experiments, each run in triplicate.

FIG. 4 shows quantification of binding of peptides to TNF-alpha by Flow cytometry. Fluorescence-Activated Cell Sorter (FACS) analysis of TNF-alpha binding to cellular receptors in presence of peptide with Sequence ID-2 and Sequence ID-6 is presented in FIG. 4a. Y-axis denotes the Mean±SE percent cells positive for TNF-RI expression of three independent experiments. Numbers 1-5 on the X axis in the bar diagram (FIG. 4a) or in the flow cytometric histogram (FIG. 4b) are as follows:

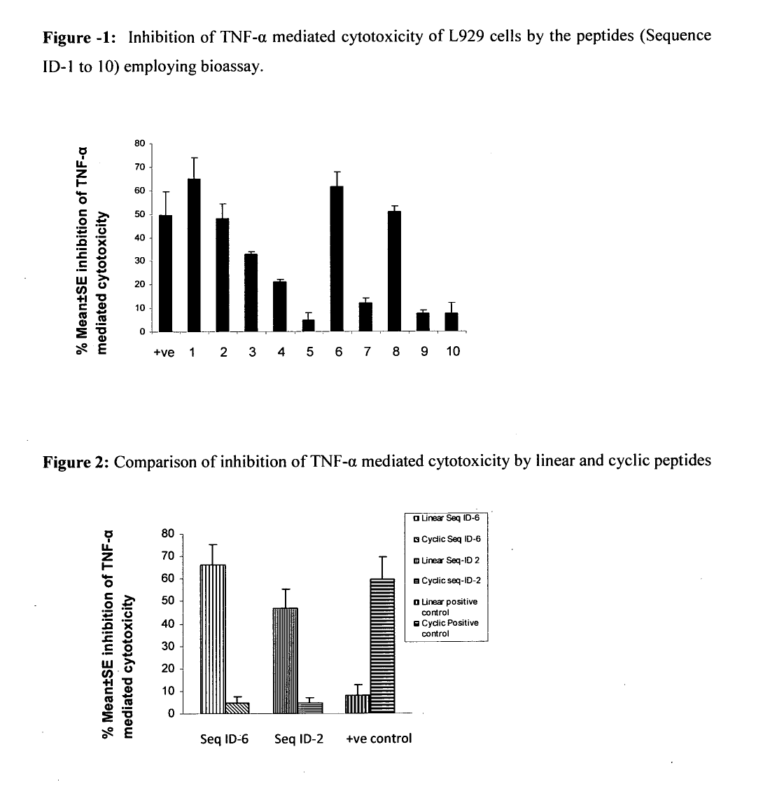

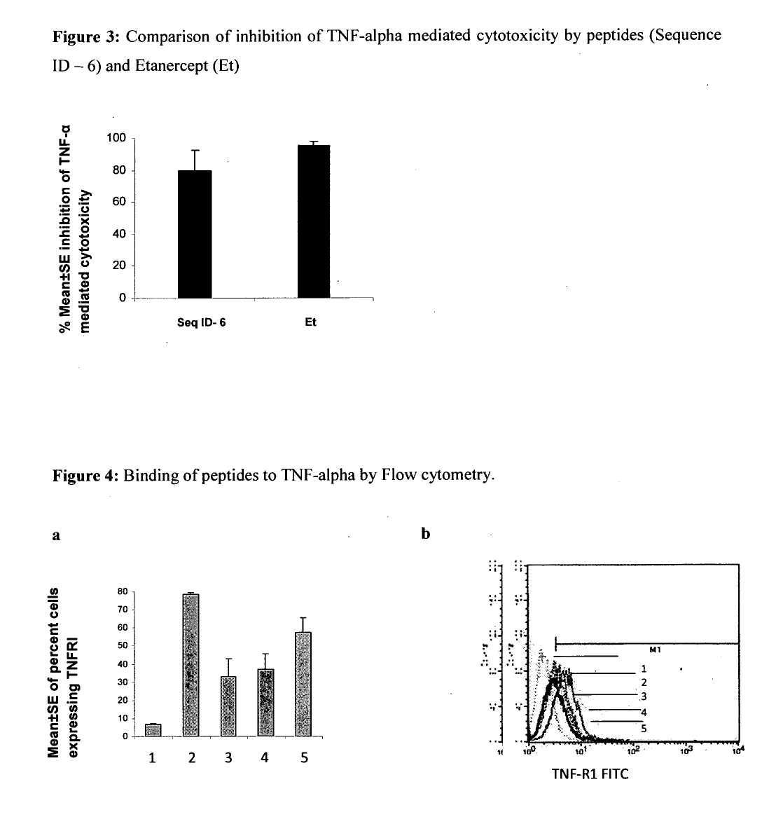

1=U937 cells stained with anti-mouse IgG FITC as secondary antibody,
2=U937 cells stained with mouse anti-human TNF-receptor antibody and anti-mouse IgG FITC
3=U937 cells treated with recombinant TNF-alpha and stained with mouse anti-human TNF-receptor antibody and anti-mouse IgG FITC,
4=U937 cells treated with a complex of recombinant TNF-alpha with sequence ID-2 and stained with mouse anti-human TNF-receptor antibody and anti-mouse IgG FITC,
5=U937 cells treated with a complex of recombinant TNF-alpha with peptide sequence ID-6 and stained with mouse anti-human TNF-receptor antibody and anti-mouse IgG FITC.

FIG. 5 shows binding of peptide of Sequence ID-6 to TNF-R1 on U937 cells: Fluorescence-Activated Cell Sorter (FACS) analysis of binding of peptide with Sequence ID-6 to TNF-R1 expressed on U937 cells is presented in FIG. 5. Y-axis denotes the Mean±SD percent cells positive for TNF-RI expression of two independent experiments. Numbers on the X-axis in the bar diagram or in the flow cytometry histogram overlay (FIG. 5) represents following samples:

1: Untreated U937 cells,
2: U937 cells+TNF-α,
3: U937 cells+peptide of Sequence ID-6.

FIG. 6 shows estimation of anti-collagen IgG levels from serum of vehicle (CFA), control (PBS) and collagen immunized mice.

FIG. 7 shows Mean of paw thickness values in right tarsal before treatment (Pre Tx) and after treatment (Post Tx) with different doses and schedule of peptide with Sequence ID-6 in murine model of collagen induced arthritis. Each group included 4-5 animals. Values on Y axis represent Mean±SE of paw thickness in respective groups. Animal groups are represented on the X axis by alphabets, namely,

A: arthritic mice treated with 1.25 mg/kg of peptide with Sequence ID-6 at a schedule of three times in a week followed by once every week for three weeks,
B: arthritic mice treated with 2.5 mg/kg of peptide with Sequence ID-6 at a schedule of three times in a week followed by once every week for three weeks,
C: arthritic mice treated with 5 mg/kg of peptide with Sequence ID-6 at a schedule of three times in a week followed by once every week for three weeks,
D: arthritic mice treated with 7.5 mg/kg of peptide with sequence ID-6 at a schedule of one dose every week for four weeks,
E: arthritic mice treated with 7.5 mg/kg of peptide with Sequence ID-6 at a schedule of one dose in first week followed by second dose after 3 weeks,
F: arthritic mice treated with PBS as control,
G: control Animals i.e. healthy male C57BL/6 mice. p values have been calculated before and after treatment. ** indicates p value<0.01, * indicates p<0.05.

FIG. 8 (a) shows Mean±SE of paw thickness values in left and right tarsal before treatment (Pre Tx) and after treatment (Post Tx) with peptide of sequence ID-6, peptide of Sequence ID-2 and Etanercept (Et). PBS treated animals are considered as untreated animals and Control denotes the healthy male C57BL/6 mice. p values have been calculated before and after treatment. ** indicates p value<0.01, * indicates p<0.05,

FIG. 8 (b) shows comparative anticollagen IgG1/IgG2a ratios after therapy in animals treated with peptide of Sequence ID-6, peptide of Sequence ID-2 and Etanercept (Et). PBS treated animals are considered as untreated animals and Control denotes the healthy male C57BL/6 mice. p values have been calculated before and after treatment. ** indicates p value<0.01, * indicates p<0.05.

FIG. 9 (a) shows mean of paw thickness values in right tarsal and right joint before (Pre Tx) and after treatment (Post Tx) with peptide sequence ID-6 and Etanercept (Et). PBS treated animals are considered as untreated animals. Each group included 3 animals.

FIG. 9 (b) shows comparative clinical score before treatment (Pre Tx) and after treatment (Post Tx) with peptide sequence ID-6 and Etanercept (Et) in rat model of adjuvant induced arthritis. PBS treated animals are considered as untreated animals. Each group included 3 animals. p values have been calculated before and after treatment. ** indicates p value<0.01, * indicates p<0.05.

FIG. 10 shows representative photographs of paw and joint swelling in arthritic rats before and after treatment with sequence ID-6, Etanercept or PBS as negative control.

### DETAILED DESCRIPTION OF THE INVENTION

The present invention relates to a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids.

The present invention also relates to a TNF-α inhibiting peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids.

The present invention further relates to biologically active peptides that act as TNF-alpha inhibitors. The biologically active peptides according to the present invention may act by different mechanisms like for example, but not limited to, following:
a) The peptides of the present invention can bind to the TNF-alpha to form a complex, resulting in prevention of binding of TNF-alpha to TNF-R1 receptors.
b) The peptides of the present invention can directly bind to the TNF-R1 receptors and can prevent TNF-alpha from binding to TNF-R1 receptors.

The present invention further relates to a pharmaceutical composition comprising a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids.

The present invention also relates to the method of treating TNF-α related disease conditions comprising administering a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids.

The present invention relates to a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids, and wherein X₁, X₂ and X₃ when taken together are not less than 2 aminoacids.

The present invention relates to a TNF-α inhibiting peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids, and wherein X₁, X₂ and X₃ when taken together are not less than 2 aminoacids.

The present invention relates to a pharmaceutical composition comprising a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids, and wherein X₁, X₂ and X₃ when taken together are not less than 2 aminoacids.

The present invention relates to the method of treating TNF-α related disease conditions comprising administering a biologically active peptide having the formula:

X₁--X₂--X₃

or pharmaceutically acceptable salts and derivatives thereof, wherein X₁, X₂ are each independently 0-2 aminoacids; X₃ is a single aminoacid residue; and aminoacids can be selected from the group comprising hydrophilic aminoacids, hydrophobic aminoacids and cysteine like aminoacids, and wherein X₁, X₂ and X₃ when taken together are not less than 2 aminoacids.

The present invention relates to a biologically active peptide having the formula:

X₁--X₂--X₃

or pharmaceutically acceptable salts and derivatives thereof, wherein X₁, X₂ are each independently 0-2 aminoacids selected from the group comprising Trp, Ser, Gln, Asn, Tyr and Leu; X₃ is a single aminoacid residue; and aminoacids can be selected from the group comprising Trp, Ser, Gln, Asn, Tyr and Leu, and wherein X₁, X₂ and X₃ when taken together are not less than 2 aminoacids.

The present invention relates to a TNF-α inhibiting peptide having the formula:

X₁--X₂--X₃

or pharmaceutically acceptable salts and derivatives thereof, wherein X₁, X₂ are each independently 0-2 aminoacids selected from the group comprising Trp, Ser, Gln, Asn, Tyr and Leu; X₃ is a single aminoacid residue; and aminoacids can be selected from the group comprising Trp, Ser, Gln, Asn, Tyr and Leu, and wherein X₁, X₂ and X₃ when taken together are not less than 2 aminoacids.

The present invention relates to a biologically active peptide having the formula:

X₁--X₂--X₃

or pharmaceutically acceptable salts and derivatives thereof, wherein X₁, X₂ and X₃ are each independently a single aminoacid residue selected from the group comprising Trp, Ser, Gln, Asn, Tyr and Leu.

The present invention relates to a TNF-α inhibiting peptide having the formula:

X₁--X₂--X₃

or pharmaceutically acceptable salts and derivatives thereof, wherein X₁, X₂ and X₃ are each independently a single aminoacid residue selected from the group comprising Trp, Ser, Gln, Asn, Tyr and Leu.

The present invention relates to a biologically active peptide having the formula:

X₁--X₂--X₃

or pharmaceutically acceptable salts and derivatives thereof, wherein X₁ is 0-2 aminoacids selected from the group comprising Trp, Ser, Gln; X₂ is 0-2 aminoacids selected from the group comprising Ser, Gln, Asn, and Tyr; and X₃ is an aminoacid residue selected from the group comprising Gln, Leu, and Tyr, and wherein X1, X2 and X3 when taken together are not less than 2 aminoacids.

The present invention relates to a TNF-α inhibiting peptide having the formula:

X₁--X₂--X₃

or pharmaceutically acceptable salts and derivatives thereof, wherein X₁ is 0-2 aminoacids selected from the group comprising Trp, Ser, Gln; X₂ is 0-2 aminoacids selected from the group comprising Ser, Gln, Asn, and Tyr; and X₃ is an aminoacid residue selected from the group comprising Gln, Leu, and Tyr, and wherein X1, X2 and X3 when taken together are not less than 2 aminoacids.

The present invention further relates to a pharmaceutical composition comprising a biologically active peptide having the formula:

X₁--X₂--X₃

or pharmaceutically acceptable salts and derivatives thereof, wherein X₁ is 0-2 aminoacids selected from the group comprising Trp, Ser, Gln; X₂ is 0-2 aminoacids selected from the group comprising Ser, Gln, Asn, and Tyr; and X₃ is an aminoacid residue selected from the group comprising Gln, Leu, and Tyr, and wherein X1, X2 and X3 when taken together are not less than 2 aminoacids; and a pharmaceutically acceptable carrier.

The present invention further relates to the method of treating TNF-α related disease conditions comprising administering a biologically active peptide having the formula:

X₁--X₂--X₃

or pharmaceutically acceptable salts and derivatives thereof, wherein X₁ is 0-2 aminoacids selected from the group comprising Trp, Ser, Gln; X₂ is 0-2 aminoacids selected from the group comprising Ser, Gln, Asn, and Tyr; and X₃ is an aminoacid residue selected from the group comprising Gln, Leu, and Tyr, and wherein X1, X2 and X3 when taken together are not less than 2 aminoacids.

The present invention further relates to a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Trp then X₂ is Ser or Gln.

The present invention further relates to a TNF-α inhibiting peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Trp then X₂ is Ser or Gln.

The present invention further relates to a pharmaceutical composition comprising a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Trp then X₂ is Ser or Gln, and a pharmaceutically acceptable carrier.

The present invention further relates to the method of treating TNF-α related disease conditions comprising administering a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Trp then X₂ is Ser or Gln.

The present invention further relates to a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Ser then X₂ is Asn or Gln.

The present invention further relates to a TNF-α inhibiting peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Ser then X₂ is Asn or Gln.

The present invention further relates to a pharmaceutical composition comprising a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Ser then X₂ is Asn or Gln, and a pharmaceutically acceptable carrier.

The present invention further relates to the method of treating TNF-α related disease conditions comprising administering a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Ser then X₂ is Asn or Gln.

The present invention further relates to a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Gln then X₂ is Asn or Tyr.

The present invention further relates to a TNF-α inhibiting peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Gln then X₂ is Asn or Tyr.

The present invention further relates to a pharmaceutical composition comprising a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Gln then X₂ is Asn or Tyr, and a pharmaceutically acceptable carrier.

The present invention further relates to the method of treating TNF-α related disease conditions comprising administering a biologically active peptide having the formula:

X₁--X₂--X₃

or pharmaceutically acceptable salts and derivatives thereof, wherein X₁ is aminoacid residue selected from the group comprising Trp, Ser, Gln; X₂ is aminoacid residue selected from the group comprising Ser, Gln, Asn, and Tyr; and X₃ is aminoacid residue selected from the group comprising Gln, Leu, and Tyr; with the proviso that if X₁ is Gln then X₂ is Asn or Tyr

As used herein, the term “peptide” refers to polymers formed by naturally occurring amino acid subunits joined by peptide bonds. The term amino acid may also refer to moieties which have portions similar to naturally occurring peptides but which have non-naturally occurring portions. Thus, peptides may have altered amino acids or linkages. The term biologically active peptide refers to the peptide which shows any kind/amount of pharmacological or biological effect when administered to mammals.

The term “aminoacid/aminoacid residues” used above may be genetically encoded L-aminoacids, naturally occurring non-genetically encoded aminoacids, synthetic L-aminoacids or D-enantiomer/s of all of the above or pharmaceutically acceptable salts/derivatives thereof. The amino acid notations used herein for the twenty genetically encoded L-amino acids and common non-encoded amino acids are conventional and are as follows:

TABLE 1

| Amino Acid | One Letter Symbol | Abbreviation |
|---|---|---|
| Alanine | A | Ala |
| Arginine | R | Arg |
| Asparagine | N | Asn |
| Aspartic acid | D | Asp |
| Cysteine | C | Cys |
| Glutamine | Q | Gln |
| Glutamic acid | E | Glu |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Trp |
| Tyrosine | Y | Tyr |
| Valine | V | Val |
| β-alanine | | bAla |
| 2,3-diaminopropionic acid | | Dpr |
| α-aminoisobutyric acid | | Aib |
| N-methylglycine (sarcosine) | | MeGly |
| Ornithine | | Orn |
| Citrulline | | Cit |
| t-butylalanine | | t-BuA |
| t-butylglycine | | t-BuG |
| N-methylisoleucine | | MeIle |
| phenylglycine | | Phg |
| cyclohexylalanine | | Cha |
| Norleucine | | Nle |
| naphthylalanine | | Nal |
| Pyridylananine | | |
| 3-benzothienyl alanine | | |
| 4-chlorophenylalanine | | Phe(4-Cl) |
| 2-fluorophenylalanine | | Phe(2-F) |
| 3-fluorophenylalanine | | Phe(3-F) |
| 4-fluorophenylalanine | | Phe(4-F) |
| Penicillamine | | Pen |
| 1,2,3,4-tetrahydro-Tic | | |
| isoquinoline-3-carboxylic | | |
| acid | | |
| β-2-thienylalanine | | Thi |
| Methionine sulfoxide | | MSO |
| Homoarginine | | hArg |
| N-acetyl lysine | | AcLys |
| 2,4-diamino butyric acid | | Dbu |
| p-aminophenylalanine | | Phe(pNH₂) |
| N-methylvaline | | MeVal |
| Homocysteine | | hCys |
| Homoserine | | hSer |
| ε-amino hexanoic acid | | Aha |
| δ-amino valeric acid | | Ava |
| 2,3-diaminobutyric acid | | Dab |

The peptides that are encompassed within the scope of the invention are partially defined in terms of amino acid residues of designated classes. The amino acids may be generally categorized into three main classes: hydrophilic amino acids, hydrophobic amino acids and Cysteine-like amino acids, depending primarily on the characteristics of the amino acid side chain. These main classes may be further divided into subclasses. Hydrophilic amino acids include amino acids having acidic, basic or polar side chains and hydrophobic amino acids include amino acids having aromatic or apolar side chains. Apolar amino acids may be further subdivided to include, among others, aliphatic amino acids.

“Hydrophobic Amino Acid” refers to an amino acid having a side chain that is uncharged at physiological pH and that is repelled by aqueous solution. Examples of genetically encoded hydrophobic amino acids include Ile, Leu and Val. Examples of non-genetically encoded hydrophobic amino acids include t-BuA.

“Aromatic Amino Acid” refers to a hydrophobic amino acid having a side chain containing at least one ring having a conjugated .pi.-electron system (aromatic group). The aromatic group may be further substituted with substituent groups such as alkyl, alkenyl, alkynyl, hydroxyl, sulfanyl, nitro and amino groups, as well as others. Examples of genetically encoded aromatic amino acids include phenylalanine, tyrosine and tryptophan. Commonly encountered non-genetically encoded aromatic amino acids include phenylglycine, 2-naphthylalanine, .beta.-2-thienylalanine, 1, 2, 3, 4-tetrahydroisoquinoline-3-carboxylic acid, 4-chlorophenylalanine, 2-fluorophenylalanine, 3-fluorophenylalanine and 4-fluorophenylalanine.

“Apolar Amino Acid” refers to a hydrophobic amino acid having a side chain that is generally uncharged at physiological pH and that is not polar. Examples of genetically encoded apolar amino acids include glycine, proline and methionine. Examples of non-encoded apolar amino acids include Cha.

“Aliphatic Amino Acid” refers to an apolar amino acid having a saturated or unsaturated straight chain, branched or cyclic hydrocarbon side chain. Examples of genetically encoded aliphatic amino acids include Ala, Leu, Val and Ile. Examples of non-encoded aliphatic amino acids include Nle.

“Hydrophilic Amino Acid” refers to an amino acid having a side chain that is attracted by aqueous solution. Examples of genetically encoded hydrophilic amino acids include Ser and Lys. Examples of non-encoded hydrophilic amino acids include Cit and hCys.

“Acidic Amino Acid” refers to a hydrophilic amino acid having a side chain pK value of less than 7. Acidic amino acids typically have negatively charged side chains at physiological pH due to loss of a hydrogen ion. Examples of genetically encoded acidic amino acids include aspartic acid (aspartate) and glutamic acid (glutamate).

“Basic Amino Acid” refers to a hydrophilic amino acid having a side chain pK value of greater than 7. Basic amino acids typically have positively charged side chains at physiological pH due to association with hydronium ion. Examples of genetically encoded basic amino acids include arginine, lysine and histidine. Examples of non-genetically encoded basic amino acids include the non-cyclic amino acids ornithine, 2,3-diaminopropionic acid, 2,4-diaminobutyric acid and homoarginine.

“Polar Amino Acid” refers to a hydrophilic amino acid having a side chain that is uncharged at physiological pH, but which has a bond in which the pair of electrons shared in common by two atoms is held more closely by one of the atoms. Examples of genetically encoded polar amino acids include asparagine and glutamine. Examples of non-genetically encoded polar amino acids include citrulline, N-acetyl lysine and methionine sulfoxide.

“Cysteine-Like Amino Acid” refers to an amino acid having a side chain capable of forming a covalent linkage with a side chain of another amino acid residue, such as a disulfide linkage. Typically, cysteine-like amino acids generally have a side chain containing at least one thiol (SH) group. Examples of genetically encoded cysteine-like amino acids include cysteine. Examples of non-genetically encoded cysteine-like amino acids include homocysteine and penicillamine.

As will be appreciated by those having skill in the art, the above classification is not absolute. Several amino acids exhibit more than one characteristic property, and can therefore be included in more than one category. For example, tyrosine has both an aromatic ring and a polar hydroxyl group. Thus, tyrosine has dual properties and can be included in both the aromatic and polar categories. Similarly, in addition to being able to form disulfide linkages, cysteine also has apolar character. Thus, while not strictly classified as a hydrophobic or apolar amino acid, in many instances cysteine can be used to confer hydrophobicity to a peptide.

Certain commonly encountered amino acids which are not genetically encoded of which the peptides and peptide analogues of the invention may be composed include, but are not limited to, .beta.-alanine (b-Ala) and other omega-amino acids such as 3-aminopropionic acid (Dap), 2,3-diaminopropionic acid (Dpr), 4-aminobutyric acid and so forth; .alpha.-aminoisobutyric acid (Aib); .epsilon.-aminohexanoic acid (Aha); .delta.-aminovaleric acid (Ava); N-methylglycine or sarcosine (MeGly); ornithine (Orn); citrulline (Cit); t-butylalanine (t-BuA); t-butylglycine (t-BuG); N-methylisoleucine (MeIle); phenylglycine (Phg); cyclohexylalanine (Cha); norleucine (Nle); 2-naphthylalanine (2-Nal); 4-chlorophenylalanine (Phe(4-Cl)); 2-fluorophenylalanine (Phe(2-F)); 3-fluorophenylalanine (Phe(3-F)); 4-fluorophenylalanine (Phe(4-F)); penicillamine (Pen); 1,2,3,4-tetrahydroisoquinoline-3-carboxylic acid (Tic); .beta.-2-thienylalanine (Thi); methionine sulfoxide (MSO); homoarginine (hArg); N-acetyl lysine (AcLys); 2,3-diaminobutyric acid (Dab); 2,3-diaminobutyric acid (Dbu); p-aminophenylalanine (Phe(pNH.sub.2)); N-methyl valine (MeVal); homocysteine (hCys) and homoserine (hSer). These amino acids also fall conveniently into the categories defined above.

The classifications of the above-described genetically encoded and non-encoded amino acids are summarized in Table 2, below. It is to be understood that Table 2 is for illustrative purposes only and does not purport to be an exhaustive list of amino acid residues which may comprise the peptides and peptide analogues described herein. Other amino acid residues which are useful for making the peptides and peptide analogues described herein can be found, e.g., in Fasman, 1989, CRC Practical Handbook of Biochemistry and Molecular Biology, CRC Press, Inc., and the references cited therein. Amino acids not specifically mentioned herein can be conveniently classified into the above-described categories on the basis of known behavior and/or their characteristic chemical and/or physical properties as compared with amino acids specifically identified.

TABLE 2

| | | |
|---|---|---|
| Classification | Genetically encoded | Genetically Non-Encoded |

Hydrophobic

| | | |
|---|---|---|
| Aromatic | F, Y, W | Phg Nal, Thi, Tic, Phe(4-Cl), |
| | | Phe(2-F), Phe(3-F), Phe(4-F), |
| | | Pyridyl Ala, Benzothienyl Ala |
| Apolar/Aliphatic | M, G, P, A, V, L, I | t-BuA, t-BuG, MeIle, Nle, |
| | | MeVal, Cha, bAla, MeGly, |
| | | Aib |

Hydrophillic

| | | |
|---|---|---|
| Acidic/Basic | D, E, H, K, R | Dpr, Orn, hArg, Phe(p-NH₂), |
| | | DBU, A₂BU |
| Polar | Q, N, S, T, Y | Cit, AcLys, MSO, hSer |
| Cysteine-Like | C | Pen, hCys, β-methyl Cys |

Preferably the biologically active peptides include, but not limited to, the following Sequences:

Sequence ID-1: Trp--Ser--Gln (WSQ)
Sequence ID--2: Trp--Ser--Leu (WSL)
Sequence ID--3: Trp--Gln--Tyr (WQY)
Sequence ID--4: Ser--Gln--Tyr (SQY)
Sequence ID--5: Ser--Gln--Leu (SQL)
Sequence ID--6: Ser--Asn--Tyr (SNY)
Sequence ID--7: Gln--Tyr--Leu (QYL)
Sequence ID--8: Gln--Asn--Tyr (QNY)
Sequence ID--9: cyclic Trp--Ser--Leu CY (cy WSL)
Sequence ID-10: cyclic Ser--Asn--Tyr CY (cy SNY)
Sequence ID-11: Trp--Gln--Gln (WQQ)
Sequence ID-12: Trp--Asn--Gln (WNQ)
Sequence ID-13: Trp--Tyr--Gln (WYQ)
Sequence ID-14: Trp--Gln--Leu (WQL)
Sequence ID-15: Trp--Asn--Leu (WNL)
Sequence ID-16: Trp--Tyr--Leu (WYL)
Sequence ID-17: Trp--Ser--Tyr (WSY)
Sequence ID-18: Trp--Asn--Tyr (WNY)
Sequence ID-19: Trp--Tyr--Tyr (WYY)
Sequence ID-20: Ser--Ser--Gln (SSQ)
Sequence ID-21: Ser--Gln--Gln (SQQ)
Sequence ID-22: Ser--Asn--Gln (SNQ)
Sequence ID-23: Ser--Ser--Leu (SSL)
Sequence ID-24: Ser--Asn--Leu (SNL)
Sequence ID-25: Ser--Tyr--Leu (SYL)
Sequence ID-26: Ser--Ser--Tyr (SSY)
Sequence ID-27: Ser--Tyr--Gln (SYQ)
Sequence ID-28: Ser--Tyr--Tyr (SYY)
Sequence ID-29: Gln--Ser--Gln (QSQ)
Sequence ID-30: Gln--Gln--Gln (QQQ)
Sequence ID-31: Gln--Asn--Gln (QNQ)
Sequence ID-32: Gln--Tyr--Gln (QYQ)
Sequence ID-33: Gln--Ser--Leu (QSL)
Sequence ID-34: Gln--Gln--Leu (QQL)
Sequence ID-35: Gln--Asn--Leu (QNL)
Sequence ID-36: Gln--Ser--Tyr (QSY)
Sequence ID-37: Gln--Gln--Tyr (QQY)
Sequence ID-38: Gln--Tyr--Tyr (QYY)

More preferably the biologically active peptides include, but not limited to, Ser--Asn--Tyr (SNY) i.e. Sequence ID-6 and Trp--Ser--Leu (WSL) i.e. Sequence ID-2.

The peptides of the present invention can be linear or cyclic, preferably the peptides are linear.

In addition, a sequence of peptide with known TNF-alpha inhibitor activity Cyclic WSQYL (cy Trp-Ser-Gln-Tyr-Leu CY) was used as positive control in evaluating TNF-α antagonistic activity in in-vitro study.

In the peptides according to the present invention, the symbol “--” between amino acid residues Xₙ generally designates a backbone interlinkage. Thus, the symbol “--” usually designates an amide linkage (—C(O)—NH). It is to be understood, however, that in all of the peptides described in the specific embodiments herein, one or more amide linkages may optionally be replaced with a linkage other than amide, preferably a substituted amide or an isostere of an amide linkage.

The term TNF-alpha or TNF-α (mentioned hereinabove or hereinafter) means the same i.e. TNF-alpha or Tumor necrosis factor-alpha.

The peptides of the present invention can be synthesized by any method known in the art. Preferably, the novel peptides of the present invention were synthesized by solid phase techniques using Fmoc Strategy on automatic peptide synthesizer (Applied Biosystems 433A Peptide Synthesizer) at 1.00 mmol scale. The peptides were assembled from C-terminus to N-terminus. Peptides were synthesized using Wang Resin. The resin employed for synthesis was Wang resin (100-200 mesh) procured from Novabiochem (Substitution 1.2 mmol/g resin).

The chemical moieties were used to protect reactive side chains of the peptides during synthesis procedure. The N-terminal amino group was protected by 9-fluorenylmethoxycarbonyl (Fmoc) group. The side chain of Leucine was used unprotected. The side chain of Tryptophan was tert-Butoxycarbonyl (Boc) protected. The side chain of Asparagine and glutamine was trityl (trt) protected. Serine and tyrosine were used with t-Butyl (tBu) protection. Cysteine was S-acetamidomethyl (Acm) protected. The first amino acid was loaded on the Wang resin using 4-dimethyaminopyridine (DMAP) and Diisopropylcarbodiimide (DIC) and followed by capping using acetic anhydride. The activating reagents used for coupling amino acids to the resin include 2-(1Hbenzotriazol-1-yl)-1,1,3,3-tetramethyluronium hexafluorophosphate/1-hydroxybenzotriazole (HBTU/HOBt) and diisopropylethylamine (DIEA). The coupling reaction was carried out in NMP. After the assembly of the peptide chain was completed the peptide-resin was washed with methanol and dried. The peptide was cleaved from resin by treatment with a cleavage mixture consisting of trifluoroacetic acid, crystalline phenol, thioanisol, ethanedithiol and de-ionized water for 2-3 hrs at room temperature. The crude peptide was obtained by precipitation with cold dry ether. The precipitate was then filtered and dissolved in water and lyophilized in Vertis freeze dryer. The resulting crude peptide was purified by preparative HPLC using a Phenomenex C18 (250×22.1) reverse phase column using a gradient of 0.1% TFA in Acetonitrile and water. The eluted fractions were reanalyzed on Analytical HPLC system (Shimadzu Corporation, Japan) using a Phenomenex C18 (250×4.6) reverse phase column. Acetonitrile was evaporated and the fractions were lyophilized to obtain the pure peptide. The identity of each peptide was confirmed by mass spectra.

The cyclic forms of the peptides were cyclized on resin using iodine in dimethylformamide (DMF). The resin was treated with six fold molar excess of Iodine in DMF with mild shaking on automated shaker. The progress of the reaction was monitored by HPLC. After completion of the reaction the resin was quenched with 0.4 M Ascorbic acid in DMF. The resin was then washed with DMF and methanol and dried in vacuo for a few minutes. The cyclized peptide was finally cleaved from the resin and analyzed by RP-HPLC. The crude peptide was purified by prep HPLC and characterized by Electrospray mass spectroscopy. The peptides of the invention have been purified by art-known techniques such as high performance liquid chromatography (HPLC), ion exchange chromatography, gel electrophoresis, affinity chromatography and the like; preferably chromatographic technique. More preferably, the peptides may be purified on a semi-preparative Shimatzu HPLC system using a RP C-18 column. The actual conditions used depend on factors like net charge, hydrophobicity, hydrophilicity etc.

The peptides of the present invention may be analysed by art-known techniques such as mass spectrometry, SDS-PAGE, isoelectric focusing, 2D-eletrophoresis, chromatography-gel Filtration (separation on basis of size), ion exchange (separation on basis of charge), sequencing, protease specificity, HPLC, X-Ray crystallography etc. More preferably the peptides of the present invention have been analyzed by mass spectrometry.

The purity of peptides can be determined by any method known in the art. The purity of the peptides was determined by reverse phase HPLC. The peptides of the present invention were analyzed for the purity. The peptides of the present invention are obtained with higher purity.

Peptides of the present invention are soluble in water, physiological buffer such as acetate buffer, phosphate buffer or PBS containing DMSO.

The TNF-alpha inhibitors according to the invention are useful for treating a pathology or condition associated with levels of TNF-alpha, in excess of the levels present in a normal healthy subject. Such pathologies include, but are not limited to: acute and chronic immune and autoimmune pathologies, such as rheumatoid arthritis, systemic lupus erythematosus, psoriasis and; sepsis syndrome, including cachexia; circulatory collapse and shock resulting from acute or chronic bacterial infection; acute and chronic parasitic or infectious processes, including bacterial, viral and fungal infections; Crohn's disease, and malignant pathologies involving TNF-alpha-secreting tumors.

### Formulation and Route of Administration

The compounds of the present invention may be administered to a subject per se or in the form of a pharmaceutical composition. Pharmaceutical compositions comprising the peptides of the present invention may be manufactured by means of conventional mixing, dissolving, granulating, dragee-making, levigating, emulsifying, encapsulating, entrapping or lyophilizing processes. Pharmaceutical compositions may be formulated in conventional manner using one or more physiologically acceptable carriers, diluents, excipients or auxiliaries which facilitate processing of the active peptides or peptide analogues into preparations which can be used pharmaceutically. Proper formulation is dependent upon the route of administration chosen.

Suitable pharmaceutical carriers are described in the most recent edition of Remington's Pharmaceutical Sciences, A. Osol, a standard reference text in this field.

The pharmaceutical compositions of the present invention may be administered by any means that enables the active agent to reach the agent's site of action in the body of a mammal. The peptides of the present invention can be administered by any route of administration known in the art. The various routes of administration includes, but not limited to, topical, parenteral, transmucosal, oral, buccal, rectal, inhalation, nasal, vaginal or sublingual.

For topical administration the peptides of the present invention may be formulated as, but not limited to, solutions, gels, ointments, creams, suspensions, or the like as are well-known in the art.

Systemic formulations include those designed for administration by injection, e.g. subcutaneous, intravenous, intramuscular, intrathecal or intraperitoneal injection, as well as those designed for transdermal, transmucosal, oral or pulmonary administration.

For injection, the peptides of the present invention may be formulated in aqueous solutions, preferably in physiologically compatible buffers such as Hank's solution, Ringer's solution, or physiological saline buffer. The solution may contain formulatory agents such as suspending, stabilizing and/or dispersing agents.

Alternatively, the peptides may be in powder form for constitution with a suitable vehicle, e.g., sterile pyrogen-free water, before use.

For transmucosal administration, penetrants appropriate to the barrier to be permeated are used in the formulation. Such penetrants are generally known in the art.

For oral administration, the peptides can be readily formulated by combining the active peptides with pharmaceutically acceptable carriers known in the art. Such carriers enable the peptides of the invention to be formulated as tablets, pills, dragees, capsules, liquids, gels, syrups, slurries, suspensions and the like, for oral ingestion by a patient to be treated.

If desired, solid dosage forms may be coated using standard techniques.

For oral liquid preparations such as, for example, suspensions, elixirs and solutions, suitable carriers, excipients or diluents include water, glycols, oils, alcohols, etc. Additionally, flavoring agents, preservatives, coloring agents and the like may be added.

For buccal administration, the peptides may take the form of tablets, lozenges, etc. formulated in conventional manner.

For administration by inhalation, the peptides for use according to the present invention are conveniently delivered in the form of an aerosol spray from pressurized packs or a nebulizer, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of e.g. gelatin for use in an inhaler or insufflator may be formulated containing a powder mix of the compound and a suitable powder base such as lactose or starch.

The peptides may also be formulated in rectal or vaginal compositions such as suppositories or retention enemas, e.g., containing conventional suppository bases such as cocoa butter or other glycerides.

In addition to the formulations described previously, the peptides may also be formulated as a depot preparation. Such long acting formulations may be administered by implantation (for example subcutaneously or intramuscularly) or by intramuscular injection. Thus, for example, the peptides may be formulated with suitable polymeric or hydrophobic materials (for example as an emulsion in an acceptable oil) or ion exchange resins, or as sparingly soluble derivatives, for example, as a sparingly soluble salt.

Alternatively, other pharmaceutical delivery systems may be employed. Liposomes and emulsions are well known examples of delivery vehicles that may be used to deliver peptides and peptide analogues of the invention. Certain organic solvents such as dimethylsulfoxide also may be employed, although usually at the cost of greater toxicity. Additionally, the peptides may be delivered using a sustained-release system. Various sustained-release materials have been established and are well known by those skilled in the art. Depending on the chemical nature and the biological stability of the therapeutic reagent, additional strategies for protein stabilization may be employed.

Pharmaceutically acceptable salts and derivatives according to the present invention are those salts and derivatives which substantially retain the biological activity of the free bases. The pharmaceutically acceptable salt and derivatives includes salts and derivatives which can be prepared according to the person skilled in the art.

The peptides of the present invention will generally be used in an amount effective to achieve the intended purpose. For use to treat or prevent TNF-associated disorders, the peptides of the present invention or pharmaceutical compositions thereof, are administered or applied in a therapeutically effective amount. By therapeutically effective amount is meant an amount effective to ameliorate or prevent the symptoms, or prolong the survival of, the patient being treated. Determination of a therapeutically effective amount is well within the capabilities of those skilled in the art, especially in light of the detailed disclosure provided herein.

The dosage administered will, of course, vary depending upon known factors such as the pharmacodynamic characteristics of the particular agent, and its mode and route of administration; age, health, and weight of the recipient; nature and extent of symptoms, kind of concurrent treatment, frequency of treatment, and the effect desired.

Initial dosages can also be estimated from in vivo data, e.g., animal models, using techniques that are well known in the art. One having ordinary skill in the art could readily optimize administration to humans based on animal data.

Dosage amount and interval may be adjusted individually to provide plasma levels of the peptides which are sufficient to maintain therapeutic effect. Usual patient dosages for administration by injection range from about 0.1 to 50 mg/kg/day, preferably from about 0.5 to 10 mg/kg/day. Typically, 1 to 10 mg per kg per day given in doses either multiple times in a day (6 times) or a dose at every alternate day or sustained release form is effective to obtain desired results. Dosage amount and interval may be adjusted individually to achieve plasma levels which are effective in ameliorating the pathological condition. Therapeutically effective serum levels may be achieved by administering multiple doses each day.

In cases of local administration or selective uptake, the effective local concentration of the peptides may not be related to plasma concentration. One, having skill in the art, will be able to optimize therapeutically effective local dosages without undue experimentation.

The amount of peptide administered will, of course, be dependent on the subject being treated, on the subject's weight, the severity of the affliction, the manner of administration and the judgment of the prescribing physician.

The therapy may be repeated intermittently while symptoms detectable or even when they are not detectable. The therapy may be provided alone or in combination with other drugs.

Throughout this application, various publications and patents are referenced with patents by number and other publications by author and year. Full citations for the publications are listed below. The disclosures of these publications and patents in their entireties are hereby incorporated by reference into this application in order to more fully describe the state of the art to which this invention pertains.

The invention has been described in an illustrative manner, and it is to be understood that the terminology which has been used is intended to be in the nature of words of description rather than of limitation.

Obviously, many modifications and variations of the present invention are possible in light of the above teachings. It is, therefore, to be understood that within the scope of the appended claims, the invention may be practiced otherwise than as specifically described.

### General Methodology and Results

### Example 1

### Process for Preparation of Peptide of Sequence ID-6

### Example 1a)

### Synthesis of Peptide of Sequence ID-6

Peptide of Sequence ID-6 was synthesized using solid phase peptide synthesis method on an automated or semi automated peptide synthesizer following Fmoc-chemistry. Wang resin was used for the synthesis. The substitution of the resin varied from 0.6 to 1.2 mmol/g. The side chain of tyrosine and serine was protected by tert-butyl group and the asparagine side chain was protected by trityl group. The loading of the first amino acid tyrosine to Wang resin was carried out using DIC/HOBt (3-5 eq) and DMAP (1-2 eq) in DMF at room temp for about 5-8 hrs. The deprotection of N-protected Fmoc group was done using 20% piperidine in DMF for 20-30 minutes. 15-20 ml of DMF was used for 1 mmol of reaction. Fmoc Asp(tBu)-OH (3-5 eq) was coupled to the deprotected tyrosine using DIC/HOBt(3-5 eq) at room temperature for about 3-4 hrs in DMF. The completion of the reaction was monitored by Kaiser test. The absence of blue color in the Kaiser test indicates the completion of the coupling reaction. The reaction mixture was further washed with DMF three times. Fmoc group was deprotected as described earlier and the reaction mixture was further washed with DMF three times. In a similar manner Fmoc-Ser(tBu)-OH was coupled to asparagine and deprotected. The average yield of the assembled peptide on solid phase was 90-95%.

Peptide Cleavage from Resin:

Reagent mixture (150 ml) containing TFA:Phenol:TIS:DIT:Water in the ratio of 82.5:5.0:2.5:5.0:5.0 was used to cleave the peptide from the resin. Resin loaded with peptide sequence ID-6 was kept in cleavage reagent under the ice cold environment for 15 min with constant stirring and then at room temperature for 2 hour with constant stirring. After the completion of reaction, mixture was filtered through sintered funnel and the peptide was precipitated by adding the cold di-ethyl-ether to the filtrate.

Precipitated peptide was filtered through the sintered funnel, dried, dissolved in water and finally freeze dried to obtain the crude peptide. The crude yield of the peptide was 85-90%.

### Purification of Peptide

The crude peptide was analyzed by analytical HPLC using acetonitrile and water as eluent. Purification of crude BRC605-1 was done on (Shimadzu) HPLC LC-8A using C-18 prep column (250×50 mm, 10μ) by isocratic elution in acetonitrile (0.1% TFA) water (0.1% TFA) mixture with a flow rate of 80-120 ml/min and a detection wavelength of 210 nm. Further the purified fraction was analyzed by analytical HPLC and desired fractions were pooled, lyophilized and characterized. The overall yield of the method was found be to be >70%.

### Characterization of Peptide

MS analysis—Characterization by mass spectral analysis of all the batches of peptide sequence ID-6 was done. The molecular mass of each batch comes out to be 383.5 (Positive mode)

Peptide sequencing—Characterization by peptide sequencing of all batches of peptide sequence ID-6 has been done. The peptide sequence of each batch confers with the actual sequence.

### Example-2

### Inhibition of TNF-Alpha Mediated Cytotoxicity of L-929 Cells by the Peptide Sequences

The peptides of the present invention were analyzed for inhibition of TNF-alpha induced cytotoxicity employing murine fibroblast cell line, L929. Addition of TNF-alpha to L929 cells (ATCC) induces cytotoxicity, which can be estimated by staining of viable cells with vital dyes like MTT (3-(4,5-Dimethylthiazol-2-yl)-2,5-diphenyltetrazolium bromide, a tetrazole) or crystal violet followed by extraction of dyes with methanol. Absorbance of extracted dye can be measured at 595 nm (Hansen et al. 1989, Journal of Immunological Methods, 119: 203-210). The assay was performed as follows:

L929 cells (maintained in Dulbecco's modified Eagle's medium supplemented with 10% FCS) were seeded at a density of 2×10⁵ cells/ml in a microtitre plates, and incubated with actinomycin D (ACT-D) at concentration of 1 μg/ml for 2 h at 37° C. and 5% CO₂. TNF-α (100 pg/ml) preincubated with 75 μM of peptide solution at 37° C. was added to L929 cells and incubated overnight at 37° C., 5% CO₂.

Cyclic WSQYL (cy Trp-Ser-Gln-Tyr-Leu) peptide was used as positive control for estimating the inhibition of TNF related cytotoxicity. TNF-α (100 pg/ml) preincubated with positive control peptide solution (75 μM) at 37° C. was added to L929 cells in a separate wells and incubated overnight at 37° C., 5% CO₂.

The live cells of test and positive control plates were then stained with 100 μl/well of 0.05% crystal violet and incubated at room temperature for 15 min., washed with PBS and kept for overnight drying at room temperature. Crystal violet was extracted from the cells by 100 μl per well of methanol and absorbance of extracted crystal violet dye was estimated at 595 nm. Percent inhibition in cytotoxicity was calculated by the following formula:

$$\frac{{OD}_{test} - {OD}_{TNF}}{{OD}_{ctrl} - {OD}_{TNF}} \times 100$$

Assessment of degree of inhibition of TNF-alpha induced cytotoxicity was done employing 75 μM concentration of peptides of the present invention.

### Example-2(a)

Peptides of Sequence IDs-1 to 10 were analyzed for inhibition of TNF-alpha induced cytotoxicity. The peptides with sequence ID-1, 2, 6 and 8 exhibited higher inhibition of TNF-alpha induced cytotoxicity when compared to the positive control. The percent inhibition of cytotoxicity induced by TNF-alpha was 65±9.2%, 48±6%, 62±6.2%, 51±2.3% (Mean±SE % inhibition, calculated on the basis of experiments each run in duplicate), respectively for peptide with sequence ID of 1, 2, 6, and 8.

### Example-2(b)

### Comparison of Ability to Inhibit TNF-Alpha Induced Cytotoxicity by Linear and Cyclic Forms of Sequence ID-2 and Sequence ID-6

Linear peptides of Sequence ID-2 and Sequence ID-6 and cyclic peptides with Sequence ID-9 and Sequence ID-10 were analyzed for comparing the ability to inhibit TNF-alpha induced cytotoxicity (using method as stated above). It was found that the linear peptides of Sequence ID-2 and ID-6 are more potent suppressors of TNF-alpha induced cytotoxicity than the cyclic forms of peptides Sequence ID-9 and ID-10. The cyclic forms of peptides of Sequence ID-9 and ID-10 exhibited minimal degree of inhibition as shown in FIG. 2.

Linear and Cyclic forms of positive control peptide i.e. Cyclic WSQYL (cy Trp-Ser-Gln-Tyr-Leu) were analyzed for comparing the ability to inhibit TNF-alpha induced cytotoxicity. Interestingly, a reverse pattern was observed i.e. the cyclic form of positive control peptide exhibited higher degree of TNF-alpha induced cytotoxicity (60±10%) whereas the linear form of positive control peptide was ineffective in inducing inhibition of TNF-alpha induced cytotoxicity (FIG. 2).

Linear peptides of Sequence ID-2 and Sequence ID-6 were found to have inhibitory potential as high as that of cyclic forms of the positive control which is a known TNF-alpha inhibitory peptide.

### Example-2(c)

### Comparison of Ability to Inhibit TNF-Alpha Induced Cytotoxicity by Peptides of Sequence ID-6 and Etanercept (Known and marketed TNF-alpha inhibitor as “Enbrel”)

Peptide of Sequence ID-6 and Etanercept were analyzed for comparing the ability to inhibit TNF-alpha induced cytotoxicity (using method as stated above in example 2). It was found that the peptide i.e. Sequence ID-6 has comparable ability to inhibit TNF-alpha induced cytotoxicity with that of Etanercept (FIG.-3).

### Binding Studies for the Peptide

Flow cytometry assay technique was used to investigate binding of peptides to TNF-α or TNF-R1 employing U937 cells (a TNF-receptor expressing human leukemia cell line). Binding of peptide to TNF-α prevents TNF-α from interacting with TNF-R1 which can be analyzed by flow cytometry. Similarly, direct binding of peptide with TNF-R1 reduces percent TNF-R1 positive cells which can be analyzed by flow cytometry.

### Example-3

### Inhibition of TNF-α Binding to its Receptor TNF-R1 on Cells by the Peptides

TNF-alpha binds to its cognate receptor TNF-R1 on U937 cells (ATCC) leading to a reduction in percent cells positive for TNF-R1. TNF-alpha binding peptides bind to TNF-alpha and prevent the interaction of TNF-alpha with TNF-R1. The percent cells positive for TNF-R1 can be quantified by staining with fluorochrome conjugated anti-TNF-R1 antibody.

Peptides of Sequence ID-2 and Sequence ID-6 were analyzed for inhibition of TNF-alpha binding to its receptor TNF-R1 on U937 cells. Inhibition of TNF-alpha binding to TNF-RI on U937 cells by peptides with sequence ID-2 or sequence ID-6 was estimated, using fluorescent activated cell sorter (FACS, FACSCALIBUR, Becton Dickinson, USA).

U937 cells (maintained in RPMI-1640 medium (Sigma Aldrich, USA), supplemented with 10% FCS were suspended in PBS containing 0.5% BSA (Bovine Serum Albumin) and 0.05% NaN₃ (binding buffer) at a density of 1×10⁵ cells per 100 μl of buffer. TNF-α (5 ng) was preincubated with peptide (Sequence ID-2 and Sequence ID-6 separately) solutions (50 μl) in PBS in separate tubes for 1 hr. at 37° C. The peptide TNF-alpha complex was then added to the U937 cells and incubated for 1 hr. at 4° C. U937 (1×10⁵) cells were incubated in a separate tube (as a parallel experiment) with TNF-alpha (5 ng) for 1 hr. at 4° C. The cells were then washed in binding buffer and 5 μl (1 mg/ml) of a human anti-mouse TNF receptor antibody was added (clone number HTR-9, Novus Biologicals), to the cells for 1 hr. at 4° C. These cells were then washed with binding buffer and stained with 10 μl (10 μg/ml) of fluorescein-conjugated goat anti-mouse IgG secondary antibody (GIBCO BRL, Gaithersburg, Md.) for 30 min. at 4° C. in dark. After two washes in binding buffer, the cells were analyzed using FACS Calibur flow cytometer (Becton Dickinson). The gates were set on the live cell population, and the degree of inhibition of TNF-α/cell binding by peptides was calculated on the basis of percent cells positive for TNF-RI expression.

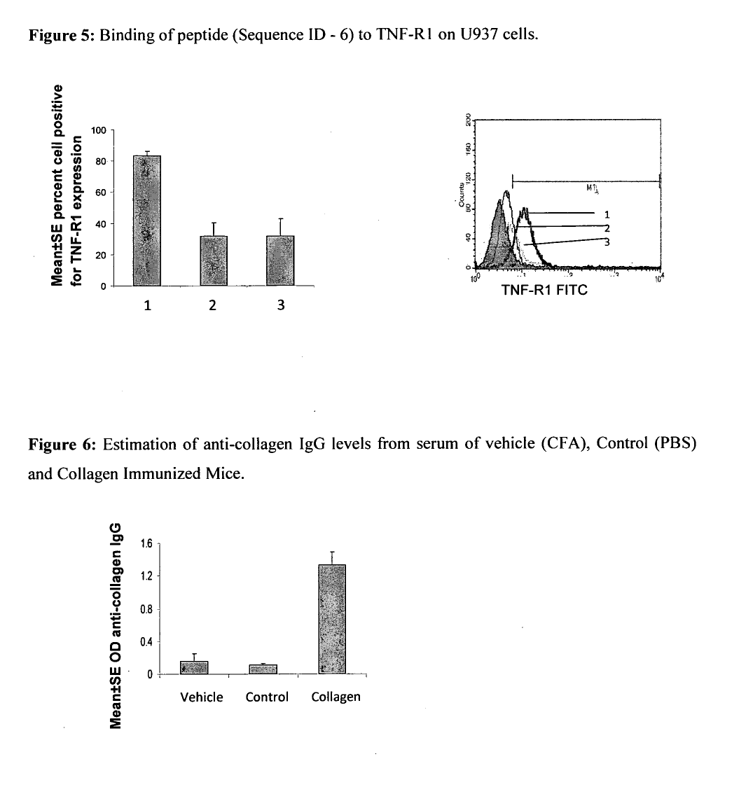

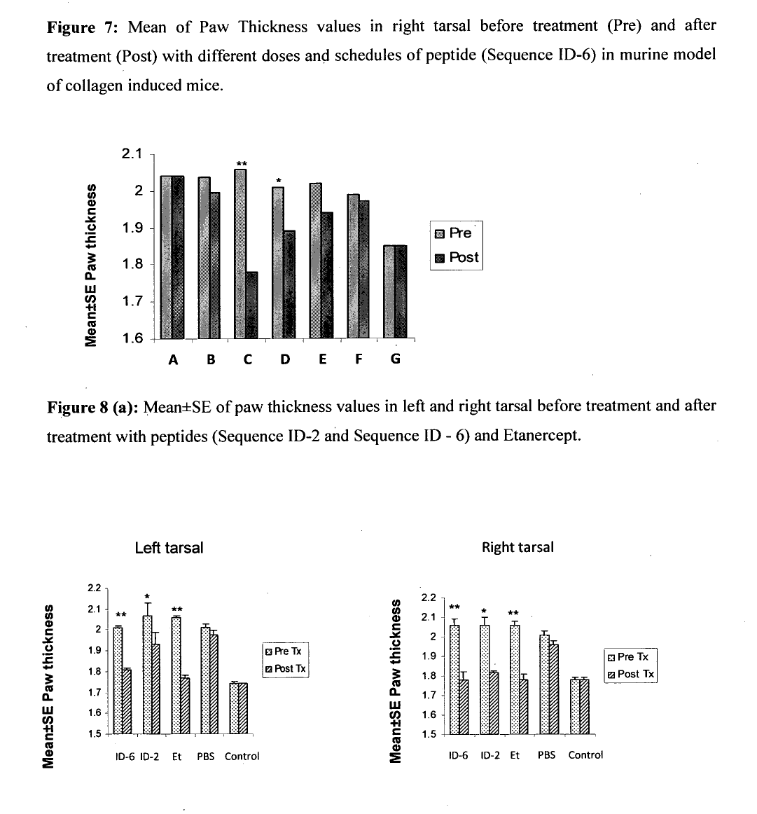

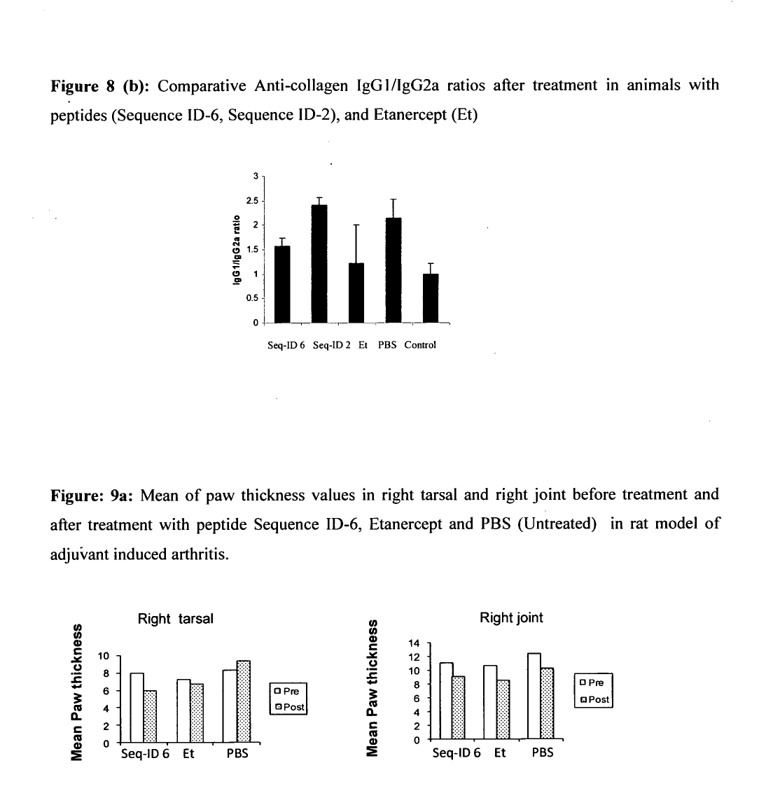

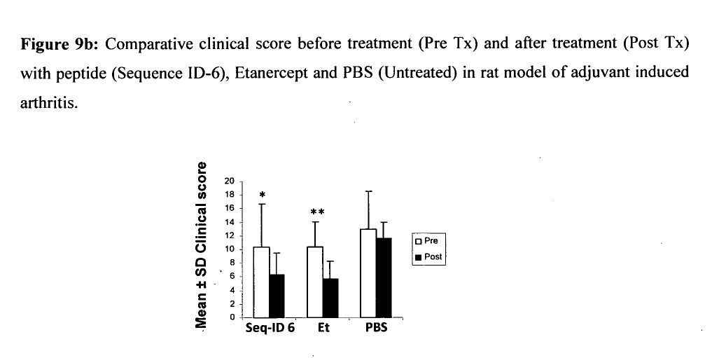

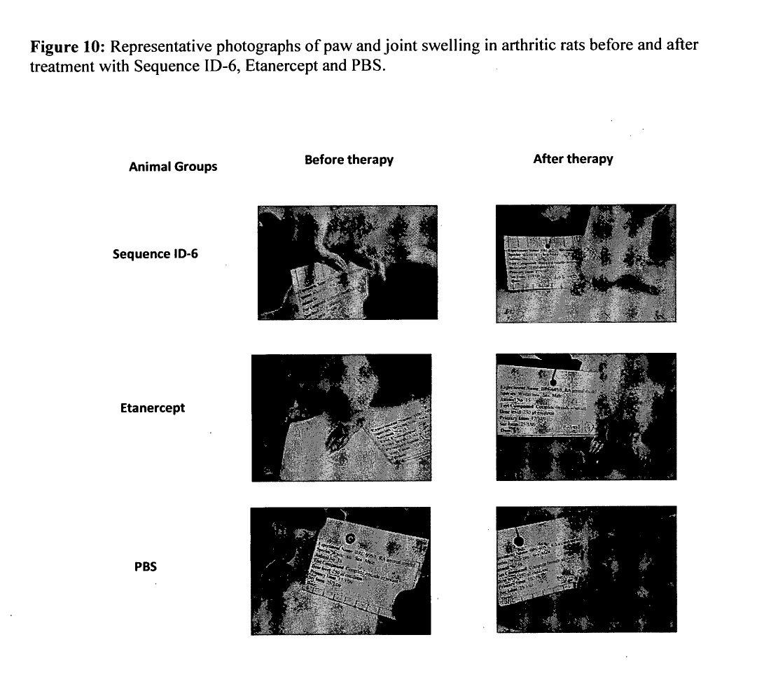

It was found that approximately 78±1% of untreated U937 cells were positive for TNF-R1 expression. Preincubation of U937 cells with TNF-alpha resulted in reduction in number of positive cells for TNF-R1 expression to 32±10% (FIGS. 4a and 4b). Preincubation of U937 cells with a complex of peptides of Sequence ID-2 and TNF-alpha resulted in resulted in number of positive cells for TNF-R1 expression to 37±8.5%. Preincubation of U937 cells with a complex of Sequence ID-6 and TNF-alpha resulted in resulted in number of positive cells for TNF-R1 expression to 57±8% (FIGS. 4a and 4b).

The above result clearly shows that the peptides of Sequence ID-2 and Sequence ID-6 binds to TNF-alpha and prevents the binding of TNF-alpha to TNF-R1 and there is less reduction in cells positive for TNF-R1 receptor when compared with the untreated TNF-alpha.

### Example-4

### Evaluation of Binding of Peptide to TNF-R1 on U937 Cells by Flow Cytometry

U937 cells were used to quantify the binding of peptides to TNF-R 1. TNF-alpha upon addition to U937 cells binds to its cognate receptor TNF-R1 on U937 cells leading to a reduction in percent cells positive for TNF-R1. TNF-alpha inhibiting peptides according to present invention bind to TNF-R1 and reduce the TNF-R1 positive cells after incubation of U937 cells with said peptides.

Peptide of Sequence ID-6 was analyzed for binding of peptide to TNF-R1 on U937 cells using flow cytometry. U937 cells were incubated with the peptide of Sequence ID-6 to demonstrate direct binding of peptide Sequence ID-6 to TNF-R1 receptor. After incubation with peptide of sequence ID-6 (250 μM) and TNF-alpha (10 ng) separately, U937 cells were washed twice and stained with 5 μl (1 mg/ml) of a human anti-mouse TNF receptor antibody (clone number HTR-9, Novus Biologicals) for 1 h at 4° C. These cells were then washed with binding buffer and stained with 10 μl (10 μg/ml) of fluorescein-conjugated goat anti-mouse IgG secondary antibody (GIBCO BRL, Gaithersburg, Md.) for 30 min. at 4° C. in dark. After two washes in binding buffer, the cells were analyzed using FACS Calibur flow cytometer (Becton Dickinson). The gates were set on the live cell population, and the degree of binding to TNF-R1 was calculated on the basis of percent cells positive for TNF-RI expression.

It was found that the expression of TNF-R1 on untreated U937 cells was 83%. Incubation of U937 cells with recombinant TNF-alpha (10 ng) resulted in reduction in percent cells positive for TNF-R1 to 31.6%. Incubation of U937 cells with peptide of Sequence ID-6 resulted in reduction in percent cells positive for TNF-R1 to 32% (FIG.-5). The reduction in percent cells positive for TNF-R1 expression after incubation with Sequence ID-6 was found comparable with that of TNF-alpha which clearly indicates binding of peptides to TNF-R1.

### Example-5

### In Vivo Efficacy of Peptides in Mouse Models of Rheumatoid Arthritis

### Example-5(a)

### Development of Mouse Model for Arthritis

Male C57BL/6 mice obtained from animal house at Lalru, Panacea Biotec were used to develop a murine model of rheumatoid arthritis. Mice were intradermally immunized with 150 μg of chicken type-II collagen (Sigma) emulsified in complete Freunds adjuvant (CFA) (Sigma). On day 17 after primary immunization, a booster immunization with 100 μg of chick type II collagen in incomplete Freunds adjuvant was administered to animals (Ethan M Shevach, Curr. Prot. Immunol. 2002: 15.0.1-15.0.6; Inglis J et al, 2008 Nature Protocols, 4:612-618). Increase in paw thickness as compared to controls (healthy, male C57BL/6 mice) and presence of anti-collagen IgG antibodies were the parameters to assess development of arthritis. Paw thickness in animals was measured using digital Vernier Calipers and anticollagen antibodies were measured in the mouse serum on day 35 after immunization.

### Example-5(b)

### Anti Collagen IgG Levels in Mouse Serum

Chicken type-II collagen (sigma) was coated onto ELISA plates at 1 μg/ml in PBS and incubated overnight at 4° C. After three washes in PBS-0.05% Tween 20 (PBS-T), serum from collagen, vehicle (CFA) and PBS (control) injected mice was added to separate wells at dilution of 1:100. Plate was incubated at room temperature for 2 h. After 5 washes in PBS-T rabbit antimouse IgG HRP (Bethyl Laboratories) was added to wells at 1:10000 dilutions and incubated at room temperature for 45 min. After washing the plate in PBS-T, OPD (ortho-phenyl diamine) as substrate was added and colour development observed. Colour development was stopped by 2N HCl. Absorbance was recorded at 490 nm (Ethan M Shevach, Cum Prot. Immunol. 2002: 15.0.1-15.0.6). Collagen immunized mice exhibited elevated serum levels of anti-collagen IgG as compared to the vehicle (CFA) and control group (PBS injected) (FIG. 6).

### Example-5(c)

### Optimization of Dose and Schedule in Murine Model

Determination of optimum dose and schedule of peptide with Sequence ID-6 was performed in a murine model of collagen induced arthritis (as prepared above). Arthritic mice were randomized according to paw thickness and divided into separate groups of 4-5 animals for intravenous administration of peptide sequence ID-6. Table-3 shows the dose and schedule of administration for different groups:

TABLE 3

| | | | No of |
|---|---|---|---|
| Group | Dose | Schedule | doses |
| A | 1.25 mg/kg | Thrice weekly followed by once every | 6 |
| | | week for three weeks | |
| B | 2.5 mg/kg | Thrice weekly followed by once every | 6 |
| | | week for three weeks | |
| C | 5 mg/kg | Thrice weekly followed by once every | 6 |
| | | week for three weeks | |
| D | 7.5 mg/kg | 1 dose every wk for 4 wks | 4 |
| E | 7.5 mg/kg | 1 dose in first wk, second dose after | 2 |
| | | 2 wks | |
| F | PBS | Thrice weekly followed by once every | |
| | | week for three weeks | |
| G | Control | Normal healthy male C57BL/6 mice | |
| | Animals | without any treatment | |

It was found that the peptides of sequence ID-6 when administered at 7.5 mg/kg once every week for 4 wks induced insignificant lowering of paw thickness in comparison to that observed in control animals. It was further observed that the peptides of sequence ID-6 when administered at a dose schedule of 5 mg/kg thrice weekly followed by once every week for 3 wks induced significant reduction of paw thickness when compared with the control animals. Therefore 5 mg/kg thrice weekly followed by once every week for 3 weeks dose was selected as the optimum dose (FIG. 7).

### Example-5(d)

### Comparative Efficacy of Peptide Sequence ID-6, ID-2 and Etanercept in Murine Model

Efficacy of peptide with sequence ID-6 and sequence ID-2 was compared with Etanercept (Marketed and approved TNF-alpha inhibiting agent with the brand name “Enbrel”) using a murine model of collagen induced arthritis (As prepared and described above). Peptides of Sequence ID-6, Sequence ID-2 and Etanercept were intravenously administered to the arthritic mice at a dose of 5 mg/kg three times in first week followed by once every week for three weeks.

It was observed that the administration of peptide with Sequence ID-6 and Etanercept resulted in significant lowering of paw thickness and was comparable to Normal control Animals (Control-healthy male C57BL/6 mice) (p<0.01) (FIG. 8a). Administration of peptide sequence ID-2 also resulted in a significant lowering of paw thickness (p<0.05) (FIG. 8a). The results clearly indicate that the peptides of Sequence ID-2 and Sequence ID-6 has efficacy comparable with that of Etanercept.

### Example 5 (e)

Comparative study for determining the IgG1/IgG2a ratios after therapy in animals treated with Peptides of Sequence ID-2, Sequence ID-6 and Etanercept.

Development of disease in collagen induced arthritis is accompanied by an increase in Th1 or proinflammatory response. IgG1/IgG2a levels were measured to determine that whether treatment with the peptides of the present invention and Etanercept improved clinical disease in murine model by decreasing Th1 response. Lower IgG1/IgG2a ratio would indicate down regulation of Th1 or proinflammatory response.

Peptides of Sequence ID-2 and Sequence ID-6, Etanercept, PBS were intravenously administered to the arthritic mice at a dose of 5 mg/kg three times in first week followed by once every week for three weeks. It was found that administration of peptides with sequence ID-6 and Etanercept resulted in lower ratio of IgG1/IgG2a after therapy as compared to untreated (PBS treated animals are considered as untreated animals) arthritic animals (FIG. 8b). Untreated arthritic animals (PBS treated animals are considered as untreated animals) exhibited higher IgG1/IgG2a ratio due to ongoing inflammation. This suggests that treatment with peptide sequence ID-6 induces therapeutic remission by decreasing Th1 response in arthritic mice and resulted in lower IgG1/IgG2a ratio, which is comparable to the IgG1/IgG2a ratio in control animals (Control-healthy male C57BL/6 mice).

### Example-6

### In Vivo Efficacy of Peptide Sequence ID-6 in Rat Model of Adjuvant Induced Arthritis

In vivo efficacy of peptide with sequence ID-6 was evaluated in a rat model of “adjuvant” induced arthritis. This model is widely used for developing and testing anti-inflammatory drugs. Adjuvant arthritis (AA) in rats mimics features of inflammatory arthritis in humans (Pearson et al, 1956, Proc. Soc. Exp. Biol. Med. 112:95-10). AA was induced in male Wistar rats by intradermal immunization with 150 μg of complete freunds adjuvant followed by a booster immunization at day 7 with incomplete freunds adjuvant. Arthritis was evaluated in animals by clinical scoring and measurement of paw and joint thickness using digital vernier calipers. The clinical score was assigned according to following criteria:

0=no erythema or swelling
1=slight erythema or swelling of one of toes or fingers
2=erythema and swelling of more than one toe or finger
3=erythema and swelling of the ankle or wrist
4=complete erythema and swelling of toes or fingers and ankle or wrist and inability to bend the ankle or wrist.

Arthritic rats were administered with peptide Sequence ID-6 at a dose of 2.5 mg/kg, once every day for 6 days in the first week followed by 3 doses every alternate day in the second week. Another group of arthritic animals received Etanercept (Et) in similar manner. Lowering of paw thickness was observed in animals that received either peptide sequence ID-6 or Etanercept as compared to Untreated arthritic rats (PBS treated rats are considered as Untreated arthritic rats) (FIG. 9a). A significant lowering of clinical score was also observed with peptide sequence ID-6 or Etanercept treatment (p<0.05) (FIG. 9b, 10). Overall, our results suggest that efficacy of peptide sequence ID-6 was comparable to Etanercept in reducing paw thickness or clinical score in arthritic rats (FIG. 9 (a) and (b).

It must be noted that as used in the specification and the appended claims, the singular forms ‘a’, ‘an’ and ‘the’ include plural references unless the context clearly indicates otherwise.

1. A TNF-α inhibiting peptide having the formula:
X₁--X₂--X₃
or pharmaceutically acceptable salts and derivatives thereof, wherein X₁, X₂ and X₃ are each independently a single amino acid residue selected from the group comprising Trp, Ser, Gln, Asn, Tyr and Leu.

2.-16. (canceled)

17. A TNF-α inhibiting peptide according to claim 1 selected from the group consisting of Sequence ID No. 1-8 and Sequence ID No. 11-38.

18. A TNF-α inhibiting peptide according to claim 17 selected from the group consisting of Sequence ID No. 1,2,6,8.

19. A process for the preparation of a TNF-α inhibiting peptide according to claim 1 wherein the peptide is prepared by solid phase synthesis.

20. A pharmaceutical composition comprising a TNF-α inhibiting peptide according to claim 1 and a pharmaceutically acceptable carrier.

21. A method of treating TNF-α related disease conditions comprising administering a TNF-α inhibiting peptide according to claim 1.

22. A pharmaceutical composition according to claim 20 wherein the composition can be administered by topical, parenteral, transmucosal, oral, buccal, rectal, inhalation, intranasal, rectal, vaginal, or sublingual route.
